(19) United States
(12) Patent Application Publication
(10) Pub. No.: US 2010/0285007 A1
(43) Pub. Date: Nov. 11, 2010

(54) DIFFERENTIAL DIAGNOSIS OF B-CELL CHRONIC LYMPHOCYTIC LEUKEMIA

(75) Inventor: Randall S. Davis, Birmingham, AL (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 12/677,754

(22) PCT Filed: Sep. 12, 2008
(86) PCT No.: PCT/US08/76182

§ 371 Date: Jun. 28, 2010

Related U.S. Application Data
(60) Provisional application No. 60/972,440, filed on Sep. 14, 2007.

(51) Int. Cl. A61K 39/395; G01N 33/68; C07K 16/18; A61P 35/02

(52) U.S. Cl. 424/133.1; 435/7.21; 436/501; 436/64; 530/387.1; 424/174.1; 530/387.3

(57) ABSTRACT

Provided are methods of diagnosing in a subject a type of B-cell chronic lymphocytic leukemia (B-CLL). Also provided are antibodies, including monoclonal antibodies, that specifically bind FCRL2, FCRL3 or FCRL5 and methods of treating B-CLL using the antibodies.

### CROSS-REFERENCE TO RELATED APPLICATIONS

This application claims priority to U.S. Provisional Application No. 60/972,440, filed Sep. 14, 2007, which is incorporated by reference herein in its entirety.

### STATEMENT REGARDING FEDERALLY FUNDED RESEARCH

This invention was made with support from Grant No. AI55638 from the National Institutes of Health. The U.S. government has certain rights in this invention.

### BACKGROUND

B cell chronic lymphocytic leukemia (B-CLL), the most prevalent leukemia in Western countries, is characterized by two subtypes that differ in the mutation status of their immunoglobulin heavy-chain variable region (IgV_{H}) gene. While patients with mutated IgV_{H} genes (MT-CLL) typically have an indolent disease course of greater than 25 years with minimal therapeutic intervention, individuals harboring unmutated IgV_{H} genes (UM-CLL) experience a more aggressive process with a median survival of eight years even after intensive treatment. Despite these differences, B-CLL cells transcriptionally and phenotypically resemble memory B lineage cells. Unfortunately, genotyping IgV_{H} regions is costly and technically challenging for most clinical laboratories, thus surrogate markers for mutation status are being investigated. Recently, expression of the Syk family tyrosine kinase ZAP-70 was found to best correlate with UM-CLL, enabling the stratification of patients into indolent or more aggressive cohorts. However, due to its cytoplasmic localization and dim expression, analysis of ZAP-70 by flow cytometry has created technical challenges resulting in diagnostic variability. Further, the expression of CD38 looked promising. CD38 expression, however, was shown to be discordant with IGHV mutations in up to 30% of cases and was shown to change over the course of the disease.

### SUMMARY

There is a need for new markers for diagnosis, subtyping, and treatment to improve the overall care of patients afflicted with B-CLL. Thus, provided are methods of diagnosing in a subject a type of B-cell chronic lymphocytic leukemia (B-CLL). For example, provided is a method comprising the steps of obtaining a biological sample from the subject, detecting in the sample one or more markers, such as FCRL3, FCRL1, FCRL2, and FCRL5. A high level of FCRL3 indicates either atypical aggressive or typical indolent B-CLL and low levels indicate typical aggressive or atypical indolent B-CLL. High levels of expression of FCRL1, FCRL2, or FCRL5 indicate indolent B-CLL and low levels indicate aggressive B-CLL. Optimally, one or more marks such as CD38, CD5, and ZAP70 are also detected in the sample. A high level of CD5 indicates atypical B-CLL and low expression indicates atypical B-CLL. A high level of CD38 indicates typical aggressive B-CLL, whereas a low level indicates typical indolent B-CLL. Also provided are antibodies, including monoclonal and humanized antibodies and fragments thereof, that specifically bind FCRL2, FCRL3 or FCRL5 and methods of treating B-CLL using the antibodies.

### DESCRIPTION OF DRAWINGS

FIG. 1 shows the specificity of a panel of human FCRL1-5-specific mAbs. FCRL1-5 mAbs were screened for cross-reactivity with BW5147 cells retrovirally transduced with plasmids expressing individual HA-tagged FCRLs. Cells were stained with the indicated biotin-coupled mouse IgG isotype FCRL mAbs (histogram, black line) or isotype matched controls (histogram, gray shade) followed by SA-PE, and analyzed by flow cytometry. Positive control staining was performed with the 12CA5 (anti-HA) mAb. FCRL1-5 mAbs also did not react with FCRL6 transductants.

FIG. 2 shows the comparison of FCRL1-5 surface expression on polyclonal B cells and malignant CLL cells. Ficoll-purified mononuclear cells isolated from the blood of a patient with CLL (CLL37; Table 2) were stained with discriminating mAbs to detect polyclonal CD19⁺CD5⁻B cells and the CD19⁺CD5⁺ leukemic CLL population in order to assess FCRL1-5, CD38, ZAP-70, and CD27 expression by multiparametric flow cytometry. Histograms reflect the staining of specific mAbs (black line) versus isotype-matched controls (gray shade) within the respective gated populations. The numbers indicated within histograms specify the MFI ratio (top values) and the percent of the population staining positive (bottom values). The MFI ratio is derived from the MFI of the test mAb divided by that of the isotype-matched control. Note that the underlined numbers in the ZAP-70 panel indicate the T/B-CLL MFI ratio.

FIGS. 3A and 3B show heterogeneous FCRL protein expression on CLL cells correlated with IGHV mutation status. FIG. 3A shows FCRL expression varies between CLL samples. Purified mononuclear cells from the blood of 2 patients with CLL, representing the UM-CLL (CLL08) and MT-CLL (CLL32) subtypes, were stained with discriminating mAbs (black line) to define FCRL1, FCRL2, FCRL3, and FCRL5 expression on the expanded CD19⁺CD5⁺CLL population. Isotype-matched control staining is indicated by histograms with a gray shade. Numerical values indicate the MFI ratio (top number) and percentage of the population staining positive (bottom number). FIG. 3B shows FCRL surface expression correlates with IGHV gene mutation status. MFI ratio comparisons determined for FCRL1, FCRL2, FCRL3, and FCRL5 expression on CD19⁺CD5⁺ cells from 55 MT-CLL samples (⋄), 52 UM-CLL samples (□), and CD19⁺B cells from 10 healthy volunteers (NL; ∘). *P<0.05 compared with UM and NL; #P<0.05 compared with NL. The characteristics of the patient samples are described in Table 2.

FIGS. 4A, 4B and 4C show FCRL2 is a stable indicator of IGHV mutation status and can predict time from diagnosis to initial treatment. FIG. 4A shows FCRL2 expression strongly correlates with the IGHV mutation status of 107 CLL samples. Cells were stained with the biotinylated 7F2 mAb or an isotype-matched control followed by SA-PE, and the FCRL2 MFI ratio was derived from the gated CD19⁺CD5⁺ population. The horizontal line indicates the 4.2 MFI ratio cutoff value. FIG. 4B shows FCRL2 surface expression on CLL cells is stable over time. MFI ratios were determined for 16 CLL samples (8 MT, F; 8 UM, E) at multiple time points (2-5) over a 39-month interval. The stability of FCRL2 expression was evaluated using the overall concordance correlation coefficient (CCC). Values greater than 0.60 suggest satisfactory stability; those greater than 0.80 indicate excellent stability based on criteria established for the K coefficient. FIG. 4C shows FCRL2, IGHV, ZAP-70 T/B-CLL ratio, and CD38 can predict time from diagnosis to initial treatment. The median time to initial therapy for patients with high FCRL2 expression (MFI ratio≧4.2) was 15.5 years compared with 3.75 years for patients with low FCRL2 expression (MFI ratio<4.2). The median time to first therapy for patients with MT-CLL was 13.5 years versus 3.51 years for patients with UM-CLL; for ZAP-70 T/B-CLL ratio of 4.0 or greater it was 13.4 years versus 4.25 years for ZAP-70 T/B-CLL ratio less than 4.0; and for CD38 less than 30%, it was 9.92 years versus 3.34 years for CD38 of 30% or greater. P values are indicated in each panel.

FIG. 5 shows FCRL3 surface expression correlates with the genotype of the 169 promoter SNP. After Ficoll separation, PBL samples from 40 normal donors were stained with anti-CD3-FITC, CD19-APC, biotinylated FCRL3 and SA-PE, or respective isotype matched controls, before cytometric analysis using a FACSCalibur™ instrument (BD Biosciences, San Jose, Calif.). MFI ratios for FCRL3 expression on CD 19+ B cells were compared with respective 169 genotypes. Mean values calculated for MFI ratios of each genotyped sample are indicated.

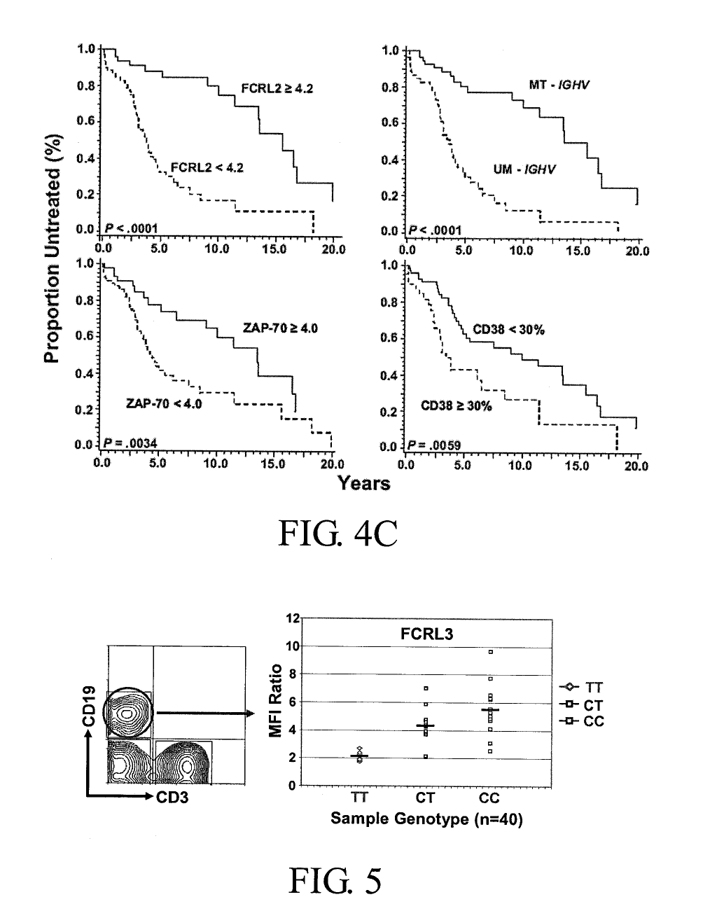

### DETAILED DESCRIPTION

B-CLL is an incurable disease characterized either by a progressive increase of anergic, self reactive, monoclonal B lineage cells that accumulate in the bone marrow and peripheral blood in a protracted fashion over many years or instead by an aggressive course which eventually manifests as bulky infiltration of lymphoid organs, progressive cellular and humoral immunodeficiency, autoimmune disease, and hematologic impotence. The clinical heterogeneity of B-CLL has become apparent over the past several years by performing gene expression profiling and examining the IgV_{H} repertoire of the B cell receptor (BCR) expressed by the leukemic clone (Stevenson and Caligaris-Cappio, Blood 103:4389-95 (2004)). Thus, patients have been stratified into two B-CLL subtypes. The more aggressive form (UM-CLL) is thought to derive from a pre-germinal center (GC) type B cell, given a lack of IgV_{H} somatic hypermutation (>98% germline sequence), while the more indolent subtype (MT-CLL) appears to originate from post-GC B lineage cells as defined by the possession of multiple IgV_{H} mutations (<98% germline) (Schroeder and Dighiero, Immunol. Today 15:288-94 (1994); Fais et al., J. Clin. Invest. 102:1515-25 (1998); and Hamblin et al., Blood 94:1848-54 (1999)). Despite these striking differences, microarray analysis by two different groups has indicated that the B-CLL transcriptome reflects an expression pattern similar to that of normal memory B cells (Rosenwald et al., J. Exp. Med. 194:1639-47 (2001); Klein et al., J. Exp. Med. 194:1625-38 (2001)). Unfortunately, sequencing IgV_{H} genes is costly, time consuming, and technically challenging. Thus, a search for surrogate markers to predict disease behavior and possibly modify treatment has been rapidly evolving. Microarray and genotyping studies have defined several other candidates that may distinguish B-CLL aggressiveness and could serve as substitutes for mutation status. The initial observation that CD38 surface expression is increased >30% in UM-CLL patients (Damle et al., Blood 94:1840-7 (1999); Hamblin et al., Blood 95:2455-7 (2000)), eventually led to discordance in subsequent studies and is now regarded as an independent poor prognostic factor (Hamblin et al., Blood 99:1023-9 (2002).

Thus far, ZAP-70, a Syk family tyrosine kinase primarily expressed in T and NK lineage cells, has been recognized as the best surrogate indicator of UM-CLL (Wiestner et al., Blood 101:4944-51 (2003); Crespo et al., N. Engl. J. Med. 348:1764-75 (2003); Orchard et al., Lancet 363:105-11 (2004)). Furthermore, in samples with discordant IgV_{H} mutation status and ZAP-70 expression, it appears to have an independent clinically significant association with disease aggressiveness (Rassenti et al., N. Engl. J. Med. 351:893-901 (2004); Del Principe et al., Blood 108:853-61 (2006)). Although it is now employed as part of the routine evaluation for B-CLL diagnosis by many clinical immunocytometry laboratories, its development as a standard marker has been complicated by its cytoplasmic localization, the differential reactivity of anti-ZAP-70 mAbs, and its variable and often dim staining intensity. This has resulted in greater technical difficulty, diagnostic inconsistency in flow cytometry results, and further complexity in assigning a cutoff threshold value (usually >10-20%) for defining a positive or negative result (Gibbs et al., Clin. Lab Haematol. 27:258-66 (2005); Titus, CAP. Today 18:1 (2004); Letestu et al., Cytometry B Clin. Cytom. 70:309-14 (2006)). An important issue for developing good diagnostic markers is establishing assays with high specificity and sensitivity using techniques that are easily applied in the clinical laboratory setting. The additional diagnostic and prognostic markers described herein can be used to subgroup B-CLL patients and to tailor their treatment options.

Unlike other B lineage malignancies where discriminating translocations involving the heavy or light chain locus provide a mechanism for perpetuating the disease, the cytogenetic aberrations in B-CLL vary. Fluorescent in situ hybridization (FISH) is useful in the detection of chromosomal aberrancies that can be predictive of an indolent disease course (13q—) versus a more aggressive one (11q— or 17p—). The B-CLL clone may also acquire additional changes as a function of time (Shanafelt et al., J. Clin. Oncol. 24:4634-41 (2006)).

The potential for an antigen-driven BCR-mediated mechanism for clonal expansion in B-CLL has been explored (Stevenson and Caligaris-Cappio, Blood 103:4389-95 (2004)). Although, most MT-CLL cells appear to be anergic given their limited ability to phosphorylate Syk after ligation of surface IgM (sIgM), these cells are still able to generate signals if sIgM is bypassed by engaging sIgD or CD79a/Iga. However, the majority of UM-CLL clones maintain signaling competence which is particularly enhanced in cells that express ZAP-70 or CD38. With the diversity of co-receptors and immunoreceptors expressed by these cells, a variety of other factors may be responsible for their signaling handicap. Alternative isoforms and somatic mutations described for CD79a/Igα and CD79b/Igβ, for example, may adversely affect B cell function. However, the underlying pathogenesis that drives the malignant transformation, differentially affects the aggressiveness of the two B-CLL subtypes.

A family of six human Fc receptor-like (FCRL) molecules and four mouse orthologs have been identified. The six human genes encode type-I glycoproteins with three to nine extracellular Ig domains of five different subtypes and cytoplasmic tails with tyrosine-based activating (ITAM or ITAM-like) or inhibitory motifs (ITIM).

The FCRL molecules are defined as close relatives of the well known Fc receptors (FcR) based on their chromosomal linkage, genomic organization, extracellular Ig domain composition, sequence homology, and tyrosine-based signaling potential. In contrast to the FcR, however, FCRL molecules possess diverse extracellular frameworks, autonomous or dual signaling properties, and except for FCRL6, which is expressed by T and NK cells, preferential B lineage expression. Most importantly, there is no compelling evidence thus far that they bind Ig or antigen-antibody complexes; thus, they remain orphan receptors.

Consensus cytoplasmic tyrosine-based motifs are highly conserved among FCRL family members. ITAMs are characterized by the sequence (E/D)-X-X-Y-X-X-(L/I)-X₆₋₈-Y-X-X-(L/I) (SEQ ID NO:1), whereas ITIMs typically have a six amino acid consensus (I/V/L/S)-X-Y-X-X-(LN) (SEQ ID NO:2). Most FCRL ITAM candidates correspond less precisely to the consensus sequence. This difference is often at the fourth position C-terminal of the tyrosine. These motifs are therefore referred to as ITAM-like. Some FCRL family members possess both ITIM and ITAM-like sequences.

Flow cytometric analysis of UM-CLL and MT-CLL samples with a panel of FCRL-specific monoclonal antibodies, indicates preferential overexpression for four of five FCRL proteins in MT-CLL (FCRL1, FCRL2, FCRL3 and FCRL5).

FCRL1-5 are preferentially expressed by B lineage cells in humans and mice. The patterns for human representatives are summarized in Table 1.

TABLE 1
Comparison of FCRL 1-5 Transcription and Protein Expression

| | | FCRL | | | | |
|---|---|---|---|---|---|---|
| | FCRL | Immunohistology | | | | |
| | Transcription | (Tonsil and | FCRL Expression by Flow Cytometry | | | |
| Receptor | (Tonsil) | Spleen) | BM | Blood | Tonsil | Spleen |
| FCRL1 | Mantle Zone | Mantle Zone (T&S) | B > Pre-B > | Pan B | N > M > | N; M |
| | | | Pro-B | | GC | |
| FCRL2 | Mantle Zone | Marginal Zone (S); | (—) | M | PC > M > N | M |
| | | Interfollicular; | | | | |
| | | Intraepithelial (T) | | | | |
| FCRL3 | GC light zone; | GC light zone; | M > | B; NK; T | M > N > | M > N; |
| | intraepithelial; | interfollicular; | Pre-B; | | GC; NK; T | NK; T |
| | intrafollicular; | intraepithelial (T) | NK; T | | | |
| | mantle zones | | | | | |
| FCRL4 | Intraepithelial; | Intraepithelial (T); | (—) | (—) | M > N | M >> N |
| | interfollicular | monocytoid B and | | | | |
| | | GC (LN) | | | | |
| FCRL5 | GC light zone; | n.d. | Pre-B; | Pan B? | PC > M > | PC > M > N |
| | intraepithelial; | | PC | | N > GC | |
| | interfollicular | | | | | |

Plasma cell (PC),
naïve (N),
memory (M),
germinal center (GC),
tonsil (T),
spleen (S),
lymph node (LN),
no data (n.d.)

FCRL1-5 transcription is uniquely detected in secondary lymphoid tissues, and, among human tonsillar B cell subpopulations, they are differentially expressed by naïve follicular mantle (IgD+CD38−), pre-GC (IgD+CD38+), GC (IgD-CD38+), memory (IgD-CD38−), and mature plasma cells (CD38++). Examination of tonsillar sections by in situ hybridization demonstrates that FCRL1-5 mark discrete cell populations within tissues. FCRL1 and FCRL2 transcripts are found within mantle zone regions, while FCRL3 and FCRL5 are expressed in the centrocyte-rich light zone of GCs, interfollicular regions, and in intraepithelial lymphocytes. A third distinct pattern is found for FCRL4 which is detected in marginal zone-like cells and intraepithelial lymphocytes.

FCRL proteins are preferentially expressed among discrete B cell populations (see Table 1). Like CD20, FCRL1 is a pan-B cell marker that peaks on naïve and memory B cells in the periphery. While FCRL2 and FCRL5 reach highest levels of expression on plasma cells and memory B cells, FCRL3 and FCRL4 appear to peak on memory B cells. In suspended tonsillar B cells, FCRL4 defines a subset of IgD-CD38—B cells that are separate from the CD27+ memory population but have the hallmark features of memory cells, IgVH region somatic hypermutation and isotype switching (Ehrhardt et al., J. Exp. Med. 202:783-91 (2005)). Except for FCRL3, FCRL1-5 expression appears to be restricted to the B lineage.

Provided herein are antibodies selective for FCRL2, FCRL3 or FCRL5. Such antibodies are useful, for example, in methods of treating and diagnosing B-CLL. Thus, provided herein are antibodies having the same epitope specificity as an antibody produced by the hybridoma cell line designated H2-7F2, which was deposited under the Budapest Treaty with the American Type Culture Collection (ATCC), Manassas, Va., on Sep. 7, 2007. The description of the deposited material was mouse hybridoma cell line expressing γ₁K isotype antibodies with the strain designation H2-7F2. The strain H2-7F2 produces FCRL2 antibodies.

Provided herein are also antibodies having the same epitope specificity as an antibody produced by the hybridoma cell line designated H3-3D2, which was deposited under the Budapest Treaty with the American Type Culture Collection (ATCC), Manassas, Va., on Sep. 7, 2007. The description of the deposited material was mouse hybridoma cell line expressing γ₁κ isotype antibodies with the strain designation H3-3D2. The strain H3-3D2 produces FCRL3 antibodies.

Provided herein are also antibodies having the same epitope specificity as an antibody produced by the hybridoma cell line designated H5-2B4, which was deposited under the Budapest Treaty with the American Type Culture Collection (ATCC), Manassas, Va., on Sep. 7, 2007. The description of the deposited material was mouse hybridoma cell line expressing γ₁κ isotype antibodies with the strain designation H5-2B4. The strain H5-2B4 produces FCRL5 antibodies.

Optionally, the antibody is produced by a cell of the hybridoma cell line deposited with the American Type Culture Collection (ATCC) as hybridoma H2-7F2, H3-3D2 or H5-2B4.

Further provided herein is a fragment of one of the described antibodies, wherein the fragment binds to an FCRL2, FCRL3 or FCRL5 receptor molecule. Optionally, the antibody or fragment activates or inhibits the FCRL2, FCRL3 or FCRL5 receptor molecule. Also provided herein is a composition comprising a one of the described antibodies or fragments that specifically bind to an FCRL2, FCRL3 or FCRL5. Optionally, the antibody fragment is selected from the group consisting of Fv, Fab, Fab′ and F(ab′)2 fragments.

The provided antibodies and fragments thereof can be utilized to modulate FCRL2, FCRL3 or FCRL5 receptor functions. An FCRL2, FCRL3 or FCRL5 receptor can be modulated by activation or inhibition of the receptor. As utilized herein, activation means that the antibody or fragment thereof binds to FCRL2, FCRL3 or FCRL5 and increases one or more receptor functions normally associated with FCRL2, FCRL3 or FCRL5. Activation does not have to be complete, as this can range from a slight increase in a receptor function to an increase similar or greater to that observed upon activation of FCRL2, FCRL3 or FCRL5 by its natural ligand. As utilized herein, inhibition means that the antibody binds to FCRL2, FCRL3 or FCRL5 and reduces one or more receptor functions normally associated with FCRL2, FCRL3 or FCRL5. Inhibition does not have to be complete, as this can range from a slight decrease in a receptor function to complete inhibition of a receptor function associated with FCRL2, FCRL3 or FCRL5. The antibodies or fragments thereof can also be used to block constitutive binding by the given receptor's ligand.

As used herein, the term antibody encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

The term variable is used herein to describe certain portions of the antibody domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

As used herein, the term epitope is meant to include any determinant capable of specific interaction with the provided antibodies. Epitopic determinants usually consist of chemically active surface groupings of molecules such as amino acids or sugar side chains and usually have specific three dimensional structural characteristics, as well as specific charge characteristics. Identification of the epitope that the antibody recognizes is performed as follows. First, various partial structures of the target molecule that the monoclonal antibody recognizes are prepared. The partial structures are prepared by synthetically preparing partial peptides of the molecule. Such peptides are prepared by, for example, known oligopeptide synthesis technique or by incorporating DNA encoding the desired partial polypeptide in a suitable expression plasmid. The expression plasmid is delivered to a suitable host, such as E. coli, to produce the peptides. For example, a series of polypeptides having appropriately reduced lengths, working from the C- or N-terminus of the target molecule, can be prepared by established genetic engineering techniques. By establishing which fragments react with the antibody, the epitope region is identified. The epitope is more closely identified by synthesizing a variety of smaller peptides or mutants of the peptides using established oligopeptide synthesis techniques. The smaller peptides are used, for example, in a competitive inhibition assay to determine whether a specific peptide interferes with binding of the antibody to the target molecule. If so, the peptide is the epitope to which the antibody binds. Commercially available kits, such as the SPOTs Kit (Genosys Biotechnologies, Inc., The Woodlands, Tex.) and a series of multipin peptide synthesis kits based on the multipin synthesis method (Chiron Corporation, Emeryvile, Calif.) may be used to obtain a large variety of oligopeptides.

The term antibody or fragments thereof can also encompass chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain FCRL2, FCRL3 or FCRL5 binding activity are included within the meaning of the term antibody or fragment thereof. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of antibody or fragments thereof are conjugates of antibody fragments and antigen binding proteins (single chain antibodies) as described, for example, in U.S. Pat. No. 4,704,692, the contents of which are hereby incorporated by reference in their entirety.

Optionally, the antibody is a monoclonal antibody. The term monoclonal antibody as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., PNAS, 81:6851-6855 (1984)).

Monoclonal antibodies may be prepared using hybridoma methods, such as those described by Kohler and Milstein, Nature, 256:495 (1975) or Harlow and Lane, Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent can be an FCRL2, FCRL3 or FCRL5 or an extracellular fragment thereof.

Generally, either peripheral blood lymphocytes (PBLs) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell lines useful here are those that fuse efficiently, support stable high level expression of antibody by the selected antibody-producing cells, and are sensitive to a medium such as HAT medium Immortalized cell lines include murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York (1987) pp. 51-63).

The culture medium in which the hybridoma cells are cultured can then be assayed for the presence of monoclonal antibodies directed against FCRL2, FCRL3 or FCRL5 or selected epitopes thereof. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art, and are described further in Harlow and Lane Antibodies, A Laboratory Manual, Cold Spring Harbor Publications, New York (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells can serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody provided herein, or can be substituted for the variable domains of one antigen-combining site of an antibody to create a chimeric bivalent antibody comprising one antigen-combining site having specificity for FCRL2, FCRL3 or FCRL5 and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen.

The Fab fragments produced in the antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or polypeptides is to link two or more peptides or polypeptides together by protein chemistry techniques. For example, peptides or polypeptides can be chemically synthesized using currently available laboratory equipment using either Fmoc (9 fluorenylmethyl-oxycarbonyl) or Boc (tert butyloxycarbonoyl) chemistry (Applied Biosystems, Inc., Foster City, Calif.). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant Ga. (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al. (1992) FEBS Lett. 307:97-101; Clark Lewis et al., J. Biol. Chem., 269:16075 (1994); Clark Lewis et al., Biochemistry, 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non peptide) bond (Schnolzer et al., Science, 256:221 (1992)). This technique has been used to synthesize analogs of protein domains as well as large amounts of relatively pure proteins with full biological activity (deLisle Milton et al., Techniques in Protein Chemistry IV. Academic Press, New York, pp. 257 267 (1992)).

The provided polypeptide fragments can be recombinant proteins obtained by cloning nucleic acids encoding the polypeptide in an expression system capable of producing the polypeptide fragments thereof, such as a bacterial, adenovirus or baculovirus expression system. For example, one can determine the active domain of an antibody from a specific hybridoma that can cause a biological effect associated with the interaction of the antibody with FCRL2, FCRL3 or FCRL5. For example, amino acids found to not contribute to either the activity or the binding specificity or affinity of the antibody can be deleted without a loss in the respective activity.

The provided fragments, whether attached to other sequences, can also include insertions, deletions, substitutions, or other selected modifications of particular regions or specific amino acids residues, provided the activity of the fragment is not significantly altered or impaired compared to the nonmodified antibody or epitope. These modifications can provide for some additional property, such as to remove or add amino acids capable of disulfide bonding, to increase its bio longevity, to alter its secretory characteristics, and the like. In any case, the fragment can possess a bioactive property, such as binding activity, regulation of binding at the binding domain, and the like. Functional or active regions may be identified by mutagenesis of a specific region of the protein, followed by expression and testing of the expressed polypeptide. Such methods are readily apparent to a skilled practitioner in the art and can include site specific mutagenesis of the nucleic acid encoding the antigen. (Zoller et al., Nucl. Acids Res. 10:6487-500 (1982).

Further provided herein is a humanized or human version of the antibody. Optionally, the antibody activates or inhibits the FCRL2, FCRL3 or FCRL5 receptor molecule. Optionally, the humanized or human antibody comprises at least one complementarity determining region (CDR) of an antibody having the same epitope specificity as an antibody produced by the hybridoma cell line deposited with the ATCC as hybridoma H2-7F2, H3-3D2 or H5-2B4. For example, the antibody can comprise all complementarity determining regions (CDRs) of an antibody having the same epitope specificity as an antibody produced by the hybridoma cell line deposited with the ATCC as hybridoma h2-7F2, H3-3D2 or H5-2B4.

Optionally, the humanized or human antibody can comprise at least one residue of the framework region of the monoclonal antibody produced by a disclosed hybridoma cell line. Humanized and human antibodies can be made using methods known to a skilled artesian; for example, the human antibody can be produced using a germ-line mutant animal or by a phage display library.

Antibodies can also be generated in other species and humanized for administration to humans. Alternatively, fully human antibodies can also be made by immunizing a mouse or other species capable of making a fully human antibody (e.g., mice genetically modified to produce human antibodies) and screening clones that bind FCRL2, FCRL3 or FCRL5. See, e.g., Lonberg and Huszar (1995) Human antibodies from transgenic mice, Int. Rev. Immunol. 13:65-93, which is incorporated herein by reference in its entirety for methods of producing fully human antibodies. As used herein, the term humanized and human in relation to antibodies, relate to any antibody which is expected to elicit a therapeutically tolerable weak immunogenic response in a human subject. Thus, the terms include fully humanized or fully human as well as partially humanized or partially human.

Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Generally, a humanized antibody has one or more amino acid residues introduced into it from a source that is non-human. These non-human amino acid residues are often referred to as import residues, which are typically taken from an import variable domain. Humanization can be essentially performed following the methods described in Jones et al., Nature, 321:522-525 (1986); Riechmann et al., Nature, 332:323-327 (1988); or Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The nucleotide sequences encoding the provided antibodies can be readily isolated and sequenced using conventional procedures (e.g., by using oligonucleotide probes that are capable of binding specifically to genes encoding the heavy and light chains of murine antibodies). These nucleotide sequences can also be modified, or humanized, for example, by substituting the coding sequence for human heavy and light chain constant domains in place of the homologous murine sequences (See U.S. Pat. No. 4,816,567 which is incorporated herein in its entirety by this reference). The nucleotide sequences encoding any of the provided antibodies can be expressed in appropriate host cells. These include prokaryotic host cells including, but not limited to, E. coli, Bacillus subtilus, other enterobacteriaceae such as Salmonella typhimurium or Serratia marcesans, and various Pseudomonas species. Eukaryotic host cells can also be utilized. These include, but are not limited to yeast cells (for example, Saccharomyces cerevisiae and Pichia pastoris), and mammalian cells such as VERO cells, HeLa cells, Chinese hamster ovary (CHO) cells W138 cells, BHK cells, COS-7 cells, 293T cells and MDCK cells. The antibodies produced by these cells can be purified from the culture medium and assayed for binding, activity, specificity or any other property of the monoclonal antibodies by utilizing the methods set forth herein and standard in the art.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., PNAS, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, ed., p. 77 (1985); Boerner et al., J. Immunol., 147(1):86-95 (1991)).

The provided antibody or fragment can be labeled or fused with another polypeptide or fragment thereof. For example, the provided antibodies or fragments thereof can be fused with a therapeutic agent. Thus, an antibody or fragment thereof that binds to FCRL2, FCRL3 or FCRL5 receptor may be linked to a therapeutic agent. The linkage can be covalent or noncovalent (e.g., ionic). Therapeutic agents include but are not limited to toxins, including but not limited to plant and bacterial toxins, small molecules, peptides, polypeptides and proteins. Genetically engineered fusion proteins, in which genes encoding for an antibody or fragments thereof, including the Fv region, can be fused to the genes encoding a toxin to deliver a toxin to the target cell are also provided. As used herein, a target cell or target cells are FCRL2, FCRL3 or FCRL5 positive cells, including for example, malignant cells of hematopoietic cell lineage, or activated or inactivated B cells.

Other examples of therapeutic agents include chemotherapeutic agents, a radiotherapeutic agent, and immunotherapeutic agent, as well as combinations thereof. In this way, the antibody complex delivered to the subject can be multifunctional, in that it exerts one therapeutic effect by binding to the extracellular domain of FCRL2, FCRL3 or FCRL5 and a second therapeutic by delivering a supplemental therapeutic agent. Binding of a monoclonal antibody to the FCRL2, FCRL3 or FCRL5 receptor can cause internalization of the receptor, which is useful for introducing a therapeutic agent such as a toxin into a cancer cell.

The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the antibody or fragment. Cleavage can be autolytic, accomplished by proteolysis, or affected by contacting the cell with a cleavage agent. Moreover, the antibody or fragments thereof can also act extracellularly, for example by initiating, affecting, enhancing or reducing an immune response without being linked in a molecular complex with a therapeutic agent. Such an antibody is known in the art as an unconjugated antibody. An unconjugated antibody can directly induce negative growth signal or apoptosis or indirectly activate a subject's defense mechanism to mediate anti-tumor activity. The antibody or fragment can be modified to enhance antibody-dependent cell killing. For example, amino acid substitutions can be made in the Fc region of the antibodies or fragments disclosed herein to increase binding of Fc receptors for enhanced antibody dependent cell cytotoxicity or increased phagocytosis. The antibody or fragment can also be used to induce cell proliferation. By inducing cell proliferation, the effects of a chemotherapeutic or radiotherapeutic agent described herein can be enhanced.

Examples of toxins or toxin moieties include diphtheria, ricin, streptavidin, and modifications thereof. An antibody or antibody fragment may be conjugated to a therapeutic moiety such as a cytotoxin, a therapeutic agent or a radioactive metal ion. A cytotoxin or cytotoxic agent includes any agent that is detrimental to cells. Examples include paclitaxel, cisplatin, carboplatin, cytochalasin B, gramicidin D, ethidium bromide, emetine, etoposide, tenoposide, colchicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil, decarbazine), alkylating agents (e.g., mechlorethamine, thiotepa, chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques for conjugating such a therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy” in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates” Immunol. Rev., 62:119-58 (1982). Alternatively, an antibody can be conjugated to a second antibody to form an antibody heteroconjugate as described by Segal in U.S. Pat. No. 4,676,980.

Further provided herein is an antibody or fragment, wherein the antibody or fragment is labeled, directly or indirectly, with a detectable moiety or marker. The detectable marker can be any marker known to those skilled in the art, or as described herein. Optionally, the detectable marker is selected from the group consisting of a fluorescent moiety, an enzyme linked moiety, a biotinylated moiety and a radiolabeled moiety. By label or detectable moiety is meant any detectable tag that can be attached directly (e.g., a fluorescent molecule integrated into a polypeptide or nucleic acid) or indirectly (e.g., by way of binding to a primary antibody with a secondary or tertiary antibody with an integrated fluorescent molecule) to the molecule of interest. Thus, a label or detectable moiety is any tag that can be visualized with imaging methods. The detectable tag can be a radio-opaque substance, a radiolabel, a fluorescent label, or a magnetic label. The detectable tag can be selected from the group consisting of gamma-emitters, beta-emitters, alpha-emitters, positron-emitters, X-ray-emitters and fluorescence-emitters suitable for localization. Suitable fluorescent compounds include fluorescein sodium, fluorescein isothiocyanate, phycoerythrin, and Texas Red sulfonyl chloride. By directly labeled is meant that the detectable moiety is attached to the antibody. By indirectly labeled is meant the detectable moiety is attached to a linker, such as, for example, a secondary or tertiary antibody.

Provided herein are methods of diagnosing a B-cell chronic lymphocytic leukemia (B-CLL). Specifically, provided are methods of diagnosing in a subject a type of B-CLL comprising obtaining a biological sample from the subject and detecting in the biological sample expression of a first marker. The first marker can be FCRL3, FCRL2, FCRL1 or FCRL5. The provided methods further comprise comparing the expression level of the first marker to a control value. When the first marker is FCRL3, a high level of FCRL3 expression as compared to a control indicates atypical aggressive B-CLL or typical indolent B-CLL in the subject. A low level of FCRL3 expression as compared to a control indicates typical aggressive B-CLL or atypical indolent B-CLL in the subject.

B-CLL is a disorder of morphologically mature but immunologically less mature lymphocytes and is manifested by progressive accumulation of these cells in the blood, bone marrow, and lymphatic tissues. B-CLL is characterized by two subtypes, indolent and aggressive. These clinical phenotypes correlate with the presence or absence of somatic mutations in the immunoglobulin heavy-chain variable region (IgV_{H}) gene. As used herein, indolent B-CLL refers to a disorder in a subjects having mutated IgV_{H} gene (MT-CLL) and/or presenting with one or more clinical phenotypes associated with indolent B-CLL. As used herein, the phrase aggressive B-CLL refers to a disorder in a subject having unmutated IgV_{H} (UM-CLL) and/or presenting with one or more clinical phenotypes associated with aggressive B-CLL. Aggressive B-CLL can be associated with overexpression of ZAP-70 (Orchard et al., Lancet 363:105-11 (2004)). Aggressive B-CLL can also be associated with expression of CD38 on ≧30% of cells (Damle et al., Blood 94:1840-7 (1999)). As used herein, atypical aggressive B-CLL or atypical indolent B-CLL is associated with low levels of expression of CD5. As used herein, typical aggressive B-CLL or atypical aggressive B-CLL is associated with high levels of expression of CD5.

Optionally, the provided methods further comprise the step of detecting in the biological sample expression of a second marker that distinguishes aggressive from indolent B-CLL and comparing the level of expression of the second marker with a control second marker value. The second marker that distinguishes aggressive from indolent B-CLL can be FCRL1, FCRL2, and FCRL5, wherein a high level of expression of FCRL1, FCRL2, or FCRL5 as compared to the control second marker value indicating indolent B-CLL and a low level of expression of FCRL1, FCRL2, or FCRL5 as compared to the control second marker value indicating aggressive B-CLL.

The expression level of FCRL1, 2, 3 or 5 can be detected by contacting the biological sample with an antibody or fragment thereof that specifically binds to the FCRL. Antibodies and fragments thereof that specifically bind to FCRL1 have been described in, for example, WO 2006/037048, which is incorporated by reference herein in its entirety. Provided herein are antibodies or fragments thereof that specifically bind to FCRL2, 3 or 5 and that are useful in the diagnostic methods.

Also provided is a method of diagnosing in a subject a type of B-cell chronic lymphocytic leukemia (B-CLL) comprising the steps of obtaining a biological sample from the subject, detecting in the biological sample expression of a first marker, wherein the first marker is FCRL2, and comparing the expression level of FCRL2 to a control FCRL2 value, a high level of FCRL2 expression compared to the control FCRL2 value indicating indolent B-CLL and a low level of expression of FCRL2 as compared to the control FCRL2 value indicating aggressive B-CLL.

Optionally, the provided methods comprise detecting in the biological sample expression of a second marker and comparing the level of the second marker with a control second marker value. The second marker can be CD19, ZAP70, CD5, CD23, CD38 or any combination thereof. The provided methods can also comprise detecting in the biological sample a third marker and so on as desired. The third marker can be CD19, ZAP70, CD5, CD23, CD38 or any combination thereof. Optionally, the third marker is different from the second marker.

For example, the provided methods optionally comprise detecting in the biological sample expression of a second marker that distinguishes typical from atypical B-CLL and comparing the level of expression of the second marker with a control second marker value. The second marker that distinguishes typical from atypical B-CLL can be CD5, wherein a high level of expression of CD5 as compared to the control second marker value indicating typical B-CLL and a low level of expression of CD5 as compared to the control second marker value indicating atypical B-CLL.

The provided methods optionally comprise detecting in the biological sample expression of a second marker that distinguishes typical indolent B-CLL from typical aggressive B-CLL and comparing expression of the second marker to a control second marker value. The second marker that distinguishes typical indolent B-CLL from typical aggressive B-CLL can be CD38 or ZAP-70, wherein a high level of CD38 or ZAP-70 expression as compared to the control second marker value indicates typical aggressive B-CLL and a low level of CD38 expression as compared to the control second marker value indicates typical indolent B-CLL.

The provided methods can also comprise detecting IgV_{H} mutational status. Such detection is performed, for example, by PCR analysis or the like.

As used herein, the phrase selectively binds, specific binding affinity, or selective for refers to a binding reaction which is determinative of the presence of, for example, a protein in a heterogeneous population of proteins, cells, proteoglycans, and other biologics. Thus, under designated conditions, the antibodies or fragments thereof bind to a protein, or protein core, epitope, fragment, or variant thereof and do not bind in a significant amount to other proteins or proteoglycans present in the subject, or in a biological sample as described herein. A significant amount of binding generally refers, for example, to an amount more than 1.5 to 2.0 times above background of the assay method.

The detecting step of the diagnostic method can be selected from methods routine in the art. Detecting can be performed using, for example, flow cytometry, or staining of preserved tissue sections or cytology. The detection step can be performed in vivo using a noninvasive medical technique such as ultrasound, computed tomography, radiography, optical coherence tomography, fluoroscopy, sonography, imaging techniques such as magnetic resonance imaging, and the like. Thus, for example, a disclosed antibody or fragment thereof, can be labeled for detection in a subject using an appropriate imaging modality. If, for example, an antibody is radiolabeled then it can be detected using radiology. Similarly, if an antibody is labeled fluorescently, then it can be detected with a light sensitive detector. In vitro detection methods can be used to detect bound antibody or fragment thereof in an ELISA, RIA, immunohistochemically, flow cytometry, FACS, 1HC, FISH, proteonomic arrays, or similar assays. The antibody, or fragment thereof, can be linked to a detectable label either directly or indirectly through use of a secondary and/or tertiary antibody; thus, bound antibody, fragment or molecular complex can be detected directly in an ELISA or similar assay.

As used herein, changes in binding refer to changes in the amount or pattern (distribution) of binding. As used throughout, a control or control value includes the level of expression in a control cell (e.g., a cell before treatment) or a control sample obtained from a subject (e.g., from the same subject before or after the effect of treatment, or from a second subject without a disorder and/or without treatment) or can comprise a known standard. The level of expression is determined, for example, from a biological sample obtained from a subject in vitro or in vivo.

As used throughout, biological sample refers to a sample from any organism. The sample can be, but is not limited to, peripheral blood, plasma, urine, saliva, gastric secretion, feces, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. The biological sample can also be whole cells or cell organelles (e.g., nuclei). A biological sample can also include a partially purified sample, cell culture, or a cell line.

Methods of treating a subject with a B-CLL are described. The methods contain the steps of administering an effective amount of an antibody or fragment thereof that specifically binds FCRL2, FCRL3 or FCRL5 to the subject. The B-CLL can be indolent B-CLL or aggressive B-CLL. The B-CLL can be typical or atypical indolent B-CLL or typical or atypical aggressive B-CLL.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, an anti-cancer compound, anti-diabetic agents, anti-inflammatory agents, anti-viral agents, anti-retroviral agents, anti-opportunistic agents, antibiotics, immunosuppressive agents, immunoglobulins, and antimalarial agents.

B-CLL treatments focus on controlling the disease and its symptoms rather than curing the disease. B-CLL is treated by chemotherapy, radiation therapy, biological therapy, allogeneic stem cell transplantation or bone marrow transplantation. Symptoms are sometimes treated surgically such as splenectomy or removal of enlarged spleen. Thus, the provided compositions can be administered in combination with chemotherapy, radiation therapy, biological therapy, allogeneic stem cell transplantation or bone marrow transplantation.

B-CLL treatments also include, but are not limited to, fludarabine, chlorambucil, fludarabine with cyclophosphamide, FCR (fludarabine, cyclophosphamide and rituximab), monoclonal antibody, alemtuzumab (directed against CD52) and CHOP (cyclophosphamide, doxorubicin, vincristine and prednisolone). Thus, the provided compositions can be administered in combination with fludarabine, chlorambucil, fludarabine with cyclophosphamide, FCR (fludarabine, cyclophosphamide and rituximab), monoclonal antibody, alemtuzumab (directed against CD52) and CHOP (cyclophosphamide, doxorubicin, vincristine and prednisolone).

The provided compositions can be administered in combination with chemotherapeutic agents. An anti-cancer compound or chemotherapeutic agent is a compound or composition effective in inhibiting or arresting the growth of an abnormally growing cell. Thus, such an agent may be used therapeutically to treat cancer as well as other diseases marked by abnormal cell growth. A pharmaceutically effective amount of an anti-cancer compound is an amount administered to an individual sufficient to cause inhibition or arrest of the growth of an abnormally growing cell. Illustrative examples of anti-cancer compounds include bleomycin, carboplatin, chlorambucil, cisplatin, colchicine, cyclophosphamide, daunorubicin, dactinomycin, diethylstilbestrol doxorubicin, etoposide, 5-fluorouracil, floxuridine, melphalan, methotrexate, mitomycin, 6-mercaptopurine, teniposide, 6-thioguanine, vincristine vinblastine, cytosine arabinoside (ara-c), thiotepa, busulfan, cytoxin, paclitaxel, methotrexate, melphalan, and carboplatin.

Any of the aforementioned treatments can be used in any combination with the compositions described herein. Combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

As used herein, references to decreasing, reducing, or inhibiting include a change of 10, 20, 30, 40, 50, 60, 70, 80, 90 percent or greater as compared to a control level. Such terms can include but do not necessarily include complete elimination.

As used herein the terms treatment, treat or treating refers to a method of reducing the effects of a disease or condition or symptom of the disease or condition. Thus in the disclosed method, treatment can refer to a 10%, 20%, 30%, 40%, 50%, 60%, 70%, 80%, 90% or 100% reduction in the severity of an established disease or condition or symptom of the disease or condition. For example, a method for treating a disease is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, administration of a therapeutic agent, that occurs before a subject begins to suffer from one or more symptoms of the disease or disorder, which inhibits or delays onset of or the severity of one or more symptoms of the disease or disorder.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Throughout this application, various publications are referenced. The disclosures of these publications in their entireties are hereby incorporated by reference into this application in order to more fully describe the state of the art to which the provided methods and compositions pertain.

A number of embodiments have been described. Nevertheless, it will be understood that various modifications may be made. Furthermore, when one characteristic or step is described it can be combined with any other characteristic or step herein even if the combination is not explicitly stated. For example, antibodies to FCRL3 may have more than one of the characteristics described herein. Similarly, any one of the method steps can be combined with other steps or with any of the antibodies described. Accordingly, other embodiments are within the scope of the claims.

The following examples are put forth so as to provide those of ordinary skill in the art with a complete disclosure and description of how the compounds, compositions, articles, and/or methods claimed herein are made and evaluated, and are intended to be purely exemplary and are not intended to limit the scope of the accompanying claims.

### EXAMPLES

### Example 1

### FCRL Expression Predicts IGHV Mutation Status and Clinical Progression in Chronic Lymphocytic Leukemia

### Materials and Methods

Recruitment of patients with CLL. CLL samples obtained from the North Shore University Hospital (NSUH) Long Island Jewish Medical Center and the Moores Cancer Center at the University of California, San Diego, met National Cancer Institute (NCl) criteria for the diagnosis of CLL, and were provided through material transfer agreements. Written informed consent was obtained from all participants before enrollment in the study in accordance with the Declaration of Helsinki, and all donor samples were anonymized to maintain health information confidentiality. Clinical characteristics, including sex, diagnosis age, diagnosis date, Rai stage, treatment history, and the date of initial therapy, were also reviewed for comparison. Although samples were derived from 3 different locations, no significant heterogeneity in the time from diagnosis to initial therapy was found among centers according to the product limit method of Kaplan-Meier and the log-rank test. Production of mAbs. FCRL-specific mAbs were generated by immunizing BALB/c mice with BW5147 mouse T-cell line transductants expressing individual hemagglutinin (HA)—tagged FCRL surface proteins (Ehrhardt et al., PNAS 100:13489-13494 (2003)), and hybridomas were produced according to standard methodology (Kearney et al., J. Immunol. 123:1548-1550 (1979)). Before subcloning, hybridoma supernatants were screened by staining FCRL1-6 stable transductants and analyzing reactivity by flow cytometry to confirm FCRL specificity and potential crossreactivity. FCRL mAb isotypes were determined by indirect capture enzyme-linked immunosorbent assay (ELISA; Zymed, San Francisco, Calif.). The anti-FCRL1 (clone 5A3; γ2bκ) mAb has been described previously (Leu et al., Blood 105:1121-6 (2005)). The anti-FCRL2 (clone 7F2; γ1κ), FCRL3 (clone 3D2; γ1κ), FCRL4 (clone 10E4; γ2bκ), and FCRL5 (clone 2B4; γ1κ) mAbs were newly generated for these studies. Purified mAbs were biotinylated using the EZ-Link Sulfo-NHS-LC-Biotin kit (Pierce, Rockford, Ill.).

Multiparameteric flow cytometry. After collection in anticoagulant containing tubes, peripheral blood mononuclear cells (PBMCs) were isolated by Ficoll-Hypaque (Mediatech, Herndon, Va.) density gradient centrifugation and either analyzed immediately or cryopreserved for future use. Mononuclear cells were stained with anti-CD5 (clone UCHT2) fluorescein isothiocyanate (FITC) and CD19-allophycocyanin (APC; clone HIB 19) to identify the expanded CLL population, costained with biotinylated anti-FCRL1-5 mAbs, and incubated with streptavidin-phycoerythrin (SA-PE) prior to 3-color flow cytometric analysis (BD Biosciences, San Diego, Calif.). Fluorochrome-conjugated isotype-matched negative control mAbs were used to detect nonspecific staining. Necrotic cells were excluded with propidium iodide (PI; Fluka, Buchs, Switzerland). After gating the lymphoid population according to typical light scatter characteristics, a total of 105 events were acquired for analysis using a FACSCalibur™ flow cytometer equipped with CellQuest™ (BD Biosciences, San Diego, Calif.) and WinMDI software (Scripps Institute, La Jolla, Calif.). To calculate relative expression levels, mean fluorescence intensity (MFI) ratios were determined by dividing the MFI of the antigen-specific fluorochrome-conjugated mAb by the MFI of the irrelevant fluorochrome-conjugated isotype-matched negative control mAb. The MFI ratio calculation assists by eliminating arbitrary assignment of the negative control mAb threshold.

CD38 surface measurements were determined with anti-CD38—PE (clone HB-7; BD Biosciences, San Diego, Calif.) according to published criteria (Damle et al., Blood 94:1840-7 (1999); Hamblin et al., Blood 99:1023-9 (2002)). A cut-off value of greater than 30% was used to determine a positive result. ZAP-70 cytoplasmic staining was performed according to comparative studies with minor modifications (Gibbs et al., Clin. Lab. Haematol. 27:258-266 (2005); Wilhelm et al., Cytometry B Cytom. 70:242-250 (2006); Rassenti et al., Cytometry B Clin. Cytom. 70:209-213 (2006); and Garff-Tavernier et al., Cytometry B Clin. Cytom. 72:103-108 (2007)). Briefly, 106 purified mononuclear cells were stained with anti-CD19-APC and CD5-FITC mAbs and incubated for 20 minutes at room temperature in the dark. Cells were then fixed and permeabilized using the Fix and Penn kit (Invitrogen, Burlingame, Calif.), counterstained with anti-ZAP-70-PE (clone 1E7.2; Invitrogen, Burlingame, Calif.) or an isotype-matched control mAb, incubated in the dark for 20 minutes, washed twice, and analyzed with a FACSCalibur™ (BD Biosciences, San Diego, Calif.) flow cytometer. A threshold of greater than 20% relative to the isotype control was considered a positive result. A second internal control-based method was also used by comparing the CD19⁻CD5⁺T-cell population with the CD19⁺CD5⁺B-CLL population. A T-cell/B-CLL cell MFI ratio less than 4 was considered a positive result (Garff-Tavernier et al., Cytometry B Clin. Cytom. 72:103-108 (2007)). IGHV gene sequencing analysis. IGHV mutation status was determined for all CLL samples according to established methods (Fais et al., J. Clin. Invest. 102:1515-1525 (1998); Campbell et al., Mol. Immunol. 29:193-203 (1992)). The IGHV sequences for CLL samples were determined in their respective laboratories (Fais et al., J. Clin. Invest. 102:1515-1525 (1998); Rassenti et al., N Engl. J. Med. 351:893-901 (2004)). Total RNA from mononuclear cell preparations was isolated using RNeasy columns (QIAGEN, Valencia, Calif.), and cDNA was synthesized with SuperScript™ III reverse transcriptase (Invitrogen, Carlsbad, Calif.) and subjected to PCR amplification using a 9700 GeneAmp® system (Applied Biosystems, Foster City, Calif.). IGHV regions were amplified with a set of six heavy-chain V_{H}-specific sense primers corresponding to the 5′ leader regions of V_{H}1 through V_{H}6 along with antisense primers complementary to human Ig constant regions (IgM, IgG, or IgA) using the KOD Hot Start DNA Polymerase (Novagen, San Diego, Calif.) (Fais et al., J. Clin. Invest. 102:1515-1525 (1998); Campbell et al., Mol. Immunol. 29:193-203 (1992)). PCR products were excised and purified using the QIAquick Gel Extraction kit (QIAGEN, Valencia, Calif.), and cloned into pBlueScript plasmids (Stratagene, Lavilla, Calif.). Inserts were sequenced on both strands by the dideoxy chain termination method using SequiTherm EXCEL™ II (Epicentre Technologies, Madison, Wis.) and an automated sequencer (LI-COR Biotechnology, Lincoln, Nebr.). Sequences from at least 6 independent clones were aligned using DNASTAR software (DNASTAR, Madison, Wis.) and compared to germline 1 g sequences in the ImMunoGeneTics (IMGT) and Joinsolver databases. A deviation less than 2% was considered unmutated (Fais et al., J. Clin. Invest. 102:1515-1525 (1998); Schroeder et al., Immunol. Today 15:288-294 (1994).

Statistical analysis. The Kruskal-Wallis test followed by the Dunn multiple comparison analysis was applied to assess FCRL expression differences among UM-CLL, MT-CLL, and normal B cells. Maximum Youden index values were used to determine optimal threshold cut-off values for prognostic factors as continuous variables based on the receiver operating characteristic (ROC) (Hanley et al., Radiology 143:29-36 (1982)).

This allowed for optimizing the cut points for outcome of CLL subtypes based on IGHV mutation status. Recursive partitioning also referred to as classification and regression tree (CART) analysis was additionally used as an alternative means for determining cutoff values. Comparisons between IGHV mutation status and the different prognostic factors were performed using univariate and multivariate logistic regression models. The CStatistic, which is a generalization of the area under the ROC curve (AUC), was used to assess the overall predictive accuracy of these models (Harrell eta 1., JAMA 247:2543-2546 (1982). The Kendall tau-b was used to quantify the strength of association between 2 variables, with values of plus or minus 0.50 indicating a strong relationship. After optimizing cut points, data assessing FCRL expression and clinical progression as defined by the time to initial therapy was estimated by constructing Kaplan-Meier survival plots and using the log-rank test. Comparisons between potential prognostic factors and time from diagnosis to initial therapy were analyzed using the Cox regression model. All statistical analysis was performed using SAS 9.1 (SAS Institute, Cary, N.C.) or the RPART Package-R version 2.5.1 (The R Foundation for Statistical Computing, Vienna, Austria).

### Results

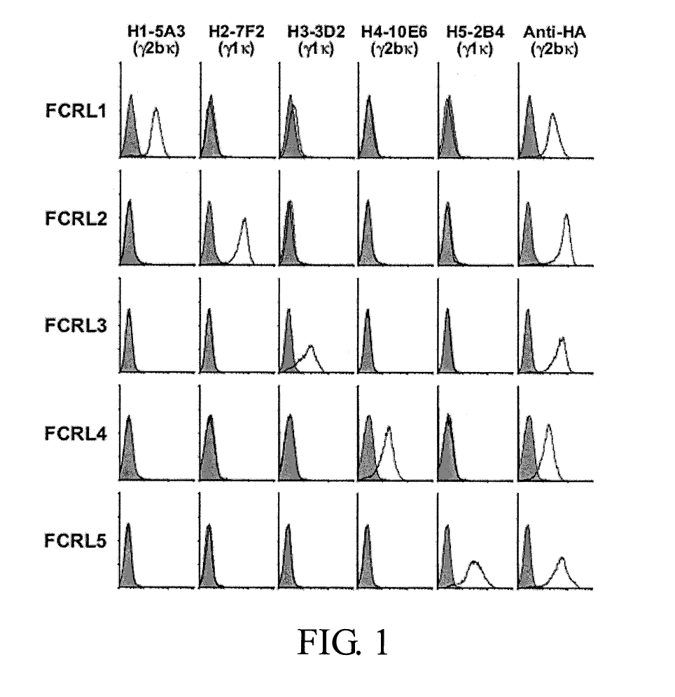

Generation of FCRL1-5-specific mAbs. A panel of FCRL-specific mAbs was generated by immunizing mice with HA-tagged retroviral transductants stably expressing human FCRL1-5 in the BW5147 mouse thymoma cell line. Reactive hybridoma subclones were screened for cross-reactivity with all 5 transductant cell lines as well as FCRL6. These mAbs were found to be receptor specific and were capable of detecting their respective proteins by immunofluorescence (FIG. 1) and immunoprecipitation.

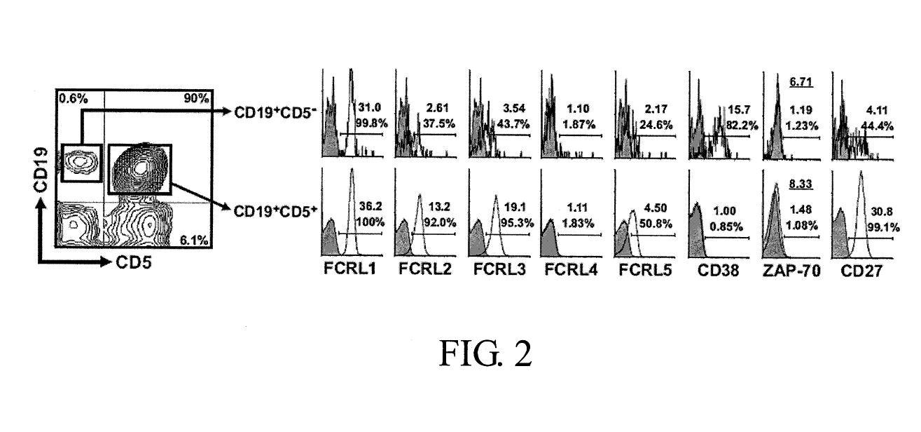

FCRL expression differs between polyclonal CD19⁺ B cells and CLL cells. Except for FCRL3, which is also identified on subsets of natural killer (NK) and T cells, FCRL1-5 are all expressed by subpopulations of human B cells. To assess whether FCRL1-5 are expressed by CLL cells, mononuclear cells prepared from the blood of a single patient were first analyzed. Staining for CD5 and CD19 identified both polyclonal CD19⁺CD5″ B cells (0.6%) and a large population of expanded CD19⁺CD5⁺CLL B cells (90%). FCRL1, FCRL2, FCRL3, and FCRL5 were detected on both the polyclonal B cells and transformed CLL cells, but FCRL4 was not (FIG. 2). In particular, FCRL1 was expressed by all blood B cells, whereas FCRL2, FCRL3, and FCRL5 were expressed by subsets of CD19⁺ cells. FCRL2, FCRL3, FCRL5, and CD27, but not FCRL1, were clearly overexpressed on the malignant CD19⁺CD5⁺B-cell population compared with polyclonal CD19⁺CD5⁻ B cells. CD38 expression was identified among the CD19⁺CD5⁻ B cells, but was not detected on the CLL clone. ZAP-70 staining was also negative in the CD19⁺ B cells and CLL cells from this donor.

An expanded analysis of 107 CLL samples and 10 age-matched healthy volunteers was then performed to determine the relative abundance of FCRL expression on malignant CLL cells versus polyclonal CD19⁺ B cells. In general, FCRL1 had the highest MFI ratios regardless of the population analyzed; however, no significant difference was found for FCRL1 expression between the entire CLL cohort and healthy donor samples or for the 3 other FCRLs by nonparametric Kruskal-Wallis test comparisons. In agreement with a prior report (Polson et al., Int. Immunol. 18:1363-1373 (2006), no significant difference in FCRL expression was found among polyclonal CD19⁺, CD19⁺CD5″, or CD19⁺CD5⁺B cells from healthy volunteers. This data indicates that FCRL1, FCRL2, FCRL3, and FCRL5 are expressed by CLL cells, but FCRL4 is not. Furthermore, FCRL expression does not significantly differ between this sample cohort of malignant CLL cells as a whole compared with normal polyclonal B cells.

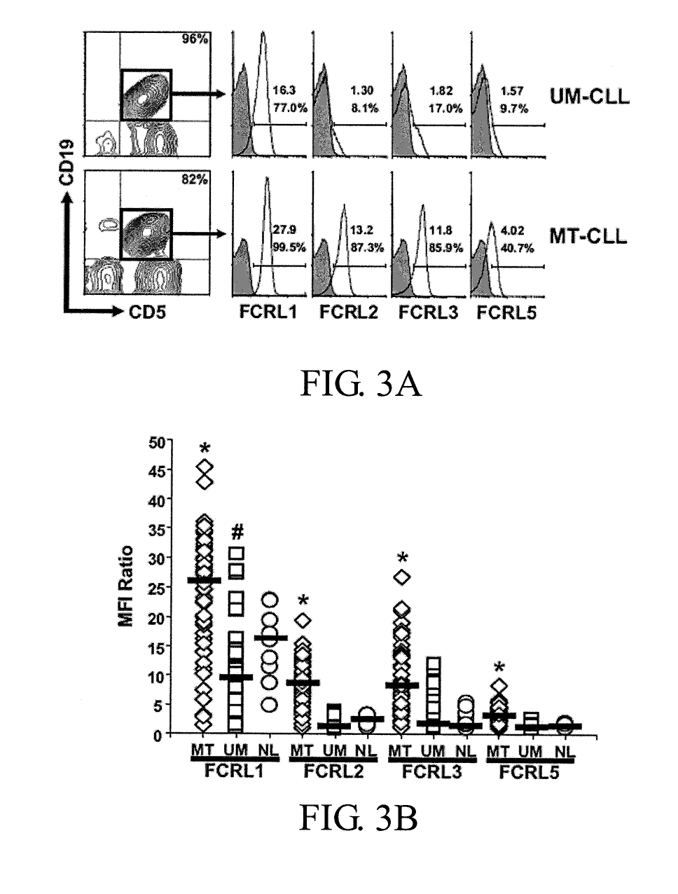

FCRL surface expression correlates with IGHV mutation status. CLL samples were next compared to determine whether FCRL surface expression varies between patients. An analysis of 2 representative patients with UM-CLL and MT-CLL that have marked differences in the surface density of FCRL proteins is depicted in FIG. 3A. While FCRL1 was expressed at relatively high levels on the leukemic expansion in both patients, FCRL5 staining was somewhat lower. In contrast, an even greater degree of difference in MFI was evident for FCRL2 and FCRL3 staining between these samples. Nevertheless, all 4 of these FCRL representatives demonstrated differences in staining between these samples, with higher levels on the MT-CLL donor relative to the UM-CLL donor.

To determine whether FCRL expression consistently correlates with the mutation status of the IGHV gene expressed by leukemic CLL cells, FCRL1, FCRL2, FCRL3, and FCRL5 surface staining on the CD19⁺CD5⁺ population from 107 IGHV genotyped CLL samples was compared with CD19⁺ polyclonal B cells from 10 healthy donors by flow cytometry. This analysis, which included 52 UM-CLL and 55 MT-CLL donors (Table 2), found that FCRL1, FCRL2, FCRL3, and FCRL5 are all expressed at significantly higher levels on MT-CLL cells compared with UM-CLL cells or polyclonal B cells from healthy volunteers (FIG. 3B).

TABLE 2
Characteristics of CLL Samples.

| | Age/ | | | | | ZAP-70 | ZAP- | | |
|---|---|---|---|---|---|---|---|---|---|
| | Sex | | | Rai | CD38 ≧ | T/B < | 70 ≧ | IGHV | Identity |
| CLL ID | Dx | Date Dx | Date Rx | Stage | 30% | 4.0 | 20% | Gene | (%) |

≧98% Germline Sequence Homology (UM-CLL) (n = 52)

| | | | | | | | | | |
|---|---|---|---|---|---|---|---|---|---|
| 03 | 50/F | September 1996 | September 1999 | II | − | + | − | 1-08 | 100 |
| 07 | 80/M | May 1990 | October 2001 | III | + | + | − | 3-64 | 99.3 |
| 08 | 64/M | March 2002 | December 2002 | IV | + | + | + | 1-69 | 100 |
| 13 | 56/M | October 1992 | January 1997 | IV | − | + | + | 1-69 | 100 |
| 21 | 68/F | February 2005 | April 2005 | 0 | − | − | − | 3-53 | 98.9 |
| 25 | 58/M | August 2005 | September 1005 | IV | + | + | + | 4-59 | 100 |
| 33 | 63/M | June 2001 | November 2005 | III | − | + | + | 3-49 | 99.7 |
| 40 | 66/M | February 2002 | November 2004 | 0 | − | − | + | 1-69 | 100 |
| 43 | 62/M | April 2004 | April 2005 | I | + | + | + | 1-69 | 99.3 |
| 45 | 58/M | April 2000 | May 2006 | II | + | + | − | 3-11 | 99.0 |
| 49 | 65/M | September 2006 | NT | III | − | + | − | 1-69 | 100 |
| 58 | 61/M | November 2004 | February 2007 | IV | + | + | + | 4-34 | 100 |
| 59 | 57/F | March 2006 | NT | 0 | + | + | − | 3-30 | 100 |
| 61 | 60/F | November 2006 | NT | 0 | + | + | + | 1-02 | 100 |
| 63 | 62/F | May 2001 | NT | 0 | − | + | + | 1-69 | 100 |
| 66 | 57/M | May 2002 | January 2007 | IV | − | + | − | 1-69 | 100 |
| 67 | 57/M | January 2007 | January 2007 | IV | + | + | − | 4-39 | 100 |
| 70 | 52/M | December 2004 | April 2007 | II | + | + | + | 1-69 | 100 |
| 72 | 83/M | April 2007 | NT | I | + | + | + | 3-11 | 100 |
| 74 | 59/M | December 2006 | NT | I | + | + | + | 1-02 | 100 |
| 77 | 67/M | January 1994 | January 1997 | II | + | + | + | 1-69 | 100 |
| 80 | 42/M | August 2003 | May 2007 | I | + | + | + | 1-69 | 99.3 |
| 83 | 50/F | April 2007 | NT | 0 | − | + | − | 4-34 | 100 |
| C569 | 52/M | January 2004 | NT | II | + | + | + | 3-21 | 100 |
| C625 | 52/M | July 2004 | May 2006 | II | + | + | + | 1-69 | 100 |
| C650 | 50/M | March 2003 | NT | I | + | + | + | 1-02 | 99.7 |
| C680 | 57/F | January 2002 | NT | I | + | + | − | 3-11 | 100 |
| C687 | 33/F | February 2002 | December 2004 | IV | + | + | + | 2-26 | 100 |
| C689 | 58/F | January 1995 | June 2003 | II | + | + | + | 3-52 | 99.3 |
| C698 | 54/F | August 2001 | June 2005 | I | − | + | + | 1-69 | 99.7 |
| C705 | 54/F | October 2003 | November 2003 | IV | + | + | − | 3-48 | 100 |
| C732 | 56/M | January 2002 | NT | II | + | − | − | 1-69 | 100 |
| C738 | 51/F | August 1997 | February 2001 | I | − | + | + | 3-33 | 100 |
| C746 | 70/M | August 2001 | August 2001 | 0 | + | + | + | 3-21 | 100 |
| C794 | 46/M | September 2002 | January 2006 | II | + | + | + | 3-23 | 100 |
| C799 | 71/M | November 2001 | December 2005 | II | − | + | + | 3-30 | 100 |
| C806 | 53/M | June 2003 | December 2005 | I | − | + | + | 1-69 | 100 |
| C810 | 51/M | June 1999 | November 2004 | I | − | + | + | 3-33 | 100 |
| C861 | 81/M | February 2006 | NT | 0 | + | + | + | 1-69 | 100 |
| C863 | 73/F | May 2005 | NT | I | + | + | + | 3-53 | 99.7 |
| C868 | 44/M | January 1998 | March 2006 | I | + | + | + | 1-69 | 100 |
| C870 | 63/F | January 2002 | April 2004 | II | − | − | − | 5-51 | 100 |
| C892 | 60/M | May 2006 | NT | I | + | + | + | 3-13 | 99.0 |
| C896 | 60/F | September 1997 | NT | 0 | + | + | + | 4-04 | 100 |
| C905 | 64/F | October 1997 | July 2001 | II | + | + | + | 2-26 | 100 |
| C907 | 55/M | January 1995 | January 1998 | II | + | + | + | 3-09 | 99.7 |
| K0011 | 36/M | June 1997 | September 1997 | I | − | + | + | 3-11 | 99.5 |
| K0024 | 77/F | January 1996 | September 1998 | II | − | + | + | 3-09 | 100 |
| K0030 | 54/F | February 1992 | August 1999 | 0 | − | + | − | 3-11 | 100 |
| K0094 | 67/M | September 1997 | November 1997 | II | − | + | + | 1-69 | 100 |
| K0095 | 50/F | January 1996 | January 1998 | II | + | + | + | 1-69 | 100 |
| K0211 | 57/M | May 1989 | October 1995 | I | + | − | + | 3-21 | 100 |

<98% Germline Sequence Homology (MT-CLL) (n = 55)

| | | | | | | | | | |
|---|---|---|---|---|---|---|---|---|---|
| 01 | 50/F | September 1996 | NT | 0 | − | − | − | 3-07 | 95.1 |
| 05 | 49/F | June 1987 | October 1998 | II | − | − | − | 4-34 | 97.0 |
| 09 | 61/M | June 1992 | NT | 0 | − | − | − | 1-69 | 91.7 |
| 10 | 69/F | February 2003 | NT | 0 | − | − | − | 4-04 | 91.8 |
| 14 | 73/F | June 1998 | NT | 0 | − | − | − | 4-34 | 88.7 |
| 15 | 72/M | September 1978 | July 1998 | IV | − | + | − | 4-34 | 96.9 |
| 16 | 55/F | June 2003 | NT | 0 | − | + | − | 3-33 | 92.4 |
| 17 | 55/M | May 2005 | NT | 0 | − | − | − | 3-53 | 94.4 |
| 20 | 59/M | March 2002 | NT | 0 | − | − | + | 3-48 | 97.4 |
| 28 | 52/M | November 1985 | May 2001 | IV | − | + | − | 1-69 | 85.1 |
| 30 | 50/F | July 1993 | October 1994 | II | − | − | − | 1-46 | 90.3 |
| 32 | 50/M | June 1995 | NT | 0 | − | − | − | 3-30 | 93.4 |
| 35 | 52/F | June 1989 | March 2006 | III | − | − | − | 4-04 | 90.9 |
| 37 | 48/M | February 2005 | NT | 0 | − | − | − | 3-23 | 90.2 |
| 44 | 62/F | November 1988 | October 1998 | 0 | − | − | − | 4-34 | 94.0 |
| 46 | 56/F | January 2001 | April 2004 | 0 | − | + | − | 4-59 | 93.3 |
| 47 | 66/F | October 1993 | September 1997 | 0 | − | − | − | 3-23 | 91.3 |
| 54 | 47/M | December 2001 | Nt | 0 | − | − | − | 4-34 | 95.4 |
| 55 | 45/m | April 2006 | Nt | I | − | − | − | 3-72 | 97.3 |
| 57 | 51/m | March 2002 | Nt | 0 | − | + | + | 3-72 | 95.2 |
| 60 | 58/F | November 2005 | NT | 0 | − | − | − | 2-05 | 95.2 |
| 62 | 41/F | April 1980 | NT | 0 | − | − | − | 4-04 | 96.9 |
| 68 | 82/F | February 2003 | NT | 0 | − | + | − | 3-23 | 93.4 |
| 75 | 51/F | May 2004 | NT | 0 | − | − | − | 4-30 | 94.5 |
| 76 | 54/M | October 2005 | NT | 0 | − | − | − | 3-72 | 91.2 |
| 78 | 60/M | December 2005 | NT | 0 | − | − | − | 4-61 | 96.2 |
| 79 | 54/F | January 1990 | August 1993 | I | − | − | − | 1-69 | 91.7 |
| 81 | 62/F | April 2007 | NT | 0 | − | − | − | 1-18 | 93.4 |
| 84 | 53/F | December 2003 | NT | I | − | − | − | 3-23 | 92.4 |
| 85 | 70/F | October 2004 | NT | 0 | − | − | − | 3-23 | 89.9 |
| C280 | 59/M | September 1999 | NT | 0 | − | + | − | 5-51 | 96.9 |
| C453 | 58/M | January 1985 | January 1986 | I | − | − | − | 3-15 | 96.0 |
| C545 | 49/F | December 2001 | NT | I | + | + | + | 3-30 | 94.6 |
| C665 | 59/F | August 2001 | NT | 0 | − | − | − | 3-21 | 94.3 |
| C678 | 56/F | September 2004 | NT | I | − | − | + | 4-04 | 95.1 |
| C712 | 44/M | January 1985 | January 1994 | I | − | − | − | 3-23 | 93.9 |
| C713 | 60/M | January 2003 | April 2005 | I | + | + | + | 4-34 | 95.9 |
| C748 | 71/M | November 2004 | NT | I | − | − | + | 1-02 | 91.5 |
| C774 | 55/M | January 1987 | July 2000 | II | − | − | − | 3-07 | 93.2 |
| C775 | 47/F | May 2005 | May 2006 | 0 | − | − | − | 3-48 | 94.6 |
| C831 | 80/F | November 2005 | NT | I | − | − | − | 3-53 | 95.6 |
| C853 | 56/M | November 2002 | April 2004 | II | + | + | + | 3-04 | 97.9 |
| C862 | 57/F | January 1982 | NT | 0 | − | − | + | 3-07 | 94.6 |
| C879 | 52/M | March 1995 | NT | 0 | − | − | − | 3-48 | 93.2 |
| C880 | 56/M | January 1997 | NT | 0 | + | + | + | 3-23 | 95.6 |
| C898 | 73/M | November 2004 | NT | I | − | + | + | 3-30 | 93.0 |
| C910 | 76/M | January 2006 | NT | 0 | − | − | − | 3-15 | 96.6 |
| K0008 | 67/M | January 1995 | April 1998 | 0 | + | + | + | 3-21 | 97.3 |
| K0103 | 77/F | December 1990 | May 2004 | II | − | − | − | 5-51 | 86.5 |
| K0107 | 70/M | November 1998 | NT | I | + | + | + | 4-39 | 97.6 |
| K0108 | 44/M | January 1985 | June 2001 | 0 | − | − | − | 3-23 | 93.3 |
| K0113 | 41/M | May 1995 | NT | 0 | − | + | + | 3-23 | 89.3 |
| K0118 | 59/M | April 1995 | NT | 0 | − | − | − | 1-69 | 94.2 |
| K0132 | 52/M | January 1993 | July 1997 | 0 | − | + | − | 3-48 | 94.7 |
| K0137 | 52/M | January 1985 | February 1990 | 0 | − | − | − | 3-30 | 86.5 |

NT: No Treatment;
C: Samples from Nicholas Chiorazzi;
K: Samples from Thomas J. Kipps.
For samples in italics and underlined, FCRL2 expression is discordant with IGHV mutation status.

While FCRL1 was expressed at higher levels on MT-CLL, the magnitude and range of the MFI ratios were much greater than those of other family members on the 3 different populations. Compared with normal CD19⁺ B cells, however, FCRL1 expression was significantly lower on UM-CLL samples. These data indicate that FCRL proteins have a heterogeneous pattern of expression in CLL that correlates with IGHV mutation status.

Elevated FCRL2 expression strongly correlates with the MT-CLL subtype. The prognostic value of FCRL1, FCRL2, FCRL3, and FCRL5 was then evaluated in the same cohort of 107 patients with CLL. The clinical characteristics of these samples are summarized in Tables 2 and 3.

TABLE 3
Clinical Characteristics of CLL Samples.

| Parameters | All Patients | UM, ≧98% | MT, <98% |
|---|---|---|---|
| No. of patients (%) | 107(100) | 52(49) | 55(51) |
| Sex, no. (%) | | | |
| Male | 64(60) | 34 | 30 |
| Female | 43(40) | 18 | 25 |
| Age at diagnosis, y | | | |
| Median | 57.0 | 58.0 | 56.0 |
| Range | 33.0-83.0 | 33.0-83.0 | 41.0-82.0 |
| Rai stage, no. (%) | | | |
| 0 | 45(42) | 10 | 35 |
| I | 29(27) | 17 | 12 |
| II | 19(18) | 14 | 5 |
| III-IV | 14(13) | 11 | 3 |
| Time to Treatment | | | |
| Median time to first | 6.4 | 3.5 | 13.5 |
| treatment, y | | | |
| No. treated (%) | 56(52) | 37 | 19 |
| No. censored events (%) | 51(48) | 15 | 36 |

This cohort of patients was typical for individuals with CLL. Among the 107 samples, the ratio of men to women was 1.5:1, with a median age of 57 years. At the time of diagnosis, 42% of patients were Rai stage 0, 27% were stage I, 18% were stage 11, and 13% were stage III or IV. A total of 52% of the samples were from patients who were treated. The median time to first therapy was 6.4 years. This analysis identified 52 patients with UM-CLL characterized by IGHV sequences 98% or more identical to the germline and 55 donors of the MT-CLL subtype defined by IGHV sequences less than 98% germline identical. The previously reported overrepresentation of the 1-69, 3-23, 4-34, and 3-07 IGHV genes frequently found among patients with CLL, and the VII 3-21 gene that has a distinct association with poor prognosis, were also observed in this analysis (Table 4).

The relationship between IGHV mutation status and FCRL expression, CD38 surface expression, and ZAP-70 cytoplasmic expression were first compared (Table 4).

TABLE 4
Correlation of IGHV mutation status with FCRL, CD38 and ZAP-70
expression.

| | | | | | | ZAP- | ZAP- |
|---|---|---|---|---|---|---|---|
| | | | | | | 70 | 70 |
| | FCRL1 | FCRL2 | FCRL3 | FCRL5 | CD38 | (T/B) | (%) |
| UM | | | | | | | |
| Median | 9.53 | 1.79 | 2.04 | 1.43 | 49.8 | 2.79 | 26.9 |
| (range) | (1.01-30.7) | (1.00-3.90) | (1.02-12.1) | (1.00-272) | (0.68-99.4) | (0.85-6.28) | (0.58-94.7) |
| Mean ± SD | 10.0 ± 7.05 | 1.93 ± 0.89 | 2.89 ± 2.36 | 1.52 ± 0.40 | 48.5 ± 36.6 | 2.82 ± 1.11 | 29.5 ± 19.1 |
| MT | | | | | | | |
| Median | 26.0 | 8.89 | 8.15 | 3.45 | 1.63 | 4.80 | 10.1 |
| (range) | (1.58-45.5) | (1.05-19.4) | (1.22-26.9) | (1.05-8.35) | (0.71-99.9) | (1.40-13.0) | (1.00-59.9) |
| Mean ± SD | 24.2 ± 9.86 | 9.07 ± 3.73 | 9.49 ± 6.03 | 3.57 ± 1.31 | 11.3 ± 26.0 | 5.24 ± 2.17 | 14.6 ± 13.1 |
| Cutoff Value | ≧15 | ≧4.2 | ≧4.0 | ≧2.3 | <30% | ≧4.0 | <20% |
| | <15 | <4.2 | <4.0 | <2.3 | ≧30% | <4.0 | ≧20% |
| UM (n = 52) | 7 | 0 | 7 | 2 | 19 | 5 | 14 |
| | 45 | 52 | 45 | 50 | 33 | 47 | 38 |
| MT (n = 55) | 45 | 49 | 46 | 49 | 49 | 39 | 42 |
| | 10 | 6 | 9 | 6 | 6 | 16 | 13 |
| Sensitivity | 81.8 | 89.1 | 83.6 | 89.1 | 89.1 | 70.9 | 76.4 |
| (%) | | | | | | | |
| Specificity | 86.5 | 100 | 86.5 | 96.2 | 63.5 | 90.4 | 73.1 |
| (%) | | | | | | | |
| PPV, % | 86.5 | 100 | 86.8 | 96.1 | 72.1 | 88.6 | 75.0 |
| NPV, % | 81.8 | 89.7 | 83.3 | 89/3 | 84.6 | 74.6 | 74.5 |
| Concordance, % | 84.1 | 94.4 | 85.0 | 92.5 | 76.6 | 80.4 | 74.8 |
| C-statistic | 0.861 | 0.964 | 0.877 | 0.946 | 0.803 | 0.864 | 0.760 |

FCRL cutoff values were determined according to the ROC and CART methods.
The indexes including sensitivity, specificity, PPV, NPV, concordance (concordance with IGHV mutation status), or C-statistic (predictive of model accuracy) were evaluated.
A C-statistic of 1.0 indicates perfect predictive discrimination, while values greater than 0.9 indicate outstanding, 0.8 to 0.9 indicate excellent, and 0.7 to 0.8 indicate fair discrimination.

In agreement with prior reports, elevated CD38 surface expression (≧30%) was significantly associated with UM-CLL samples (P<0.001), and 25 (23%) of 107 patients were similarly found to be discordant.

Methods for assessing ZAP-70 protein expression and the use and interpretation of controls vary among laboratories internationally. Based on these studies, 2 methods for measuring ZAP-70 expression were performed. In this study, the concordance rates for ZAP-70 expression and /GHVmutation status were 74.8% using the 20% and greater cutoff and 80.4% using a T/B-CLL ratio of less than 4; both results are consistent with previous findings.

After defining the clinical and prognostic characteristics of the samples, these parameters were compared with FCRL1, FCRL2, FCRL3, and FCRL5 staining results obtained by flow cytometric analysis (Table 4). MFI ratio cutoff values for the different FCRLs were determined by ROC— and CART-based analyses. A significant correlation among the expression patterns of the 4 FCRL proteins was identified. As indicated by their mean and median values, and from the results obtained in FIG. 3B, in general, FCRL proteins were expressed at higher levels on MT-CLL samples and lower levels on UM-CLL samples. The concordance in expression patterns among the 4 FCRL molecules was 77% (i.e., all FCRL representatives were either up-regulated or down-regulated on the expanded CLL population regardless of IGHV subtype). Furthermore, among the 23% of patients that differed in FCRL1, FCRL2, FCRL3, and FCRL5 expression, 18% were discordant in the expression of only a single FCRL molecule. This study found that the 4 FCRL proteins had concordance values of 84.1% to 94.4% with IGHV gene mutation status. This exceeded the association observed for CD38 and ZAP-70 expression, regardless of which staining method was used.

To evaluate the statistical significance of these different variables, univariate logistic regression was initially performed. By this analysis, 8 significant prognostic factors, including FCRL1, FCRL2, FCRL3, and FCRL5; CD38; ZAP-70 T/B-CLL ratio; ZAP-70 percentage; and Rai stage, were identified that could distinguish patients with MT-CLL from those with UM-CLL (all were P<0.001); however, both age and sex did not segregate these subgroups. Note that with this particular cohort of 107 patients with CLL, using both ROC- and CART-based analyses, it was found that a cutoff value of 13% for CD38 was slightly better in predicting IGHV mutation status, resulting in 81% concordance. These studies verified the utility of the 20% cut-off by both statistical methods using the ZAP-70 percentage technique; however, a value of 3.6 was slightly better for predicting IGHV mutation status according to the ZAP-70 T/B-CLL ratio scheme, yielding 83% concordance. When these same 10 parameters were considered by multivariate logistic analysis with stepwise selection, only FCRL2 maintained a significant association with IGHV mutation status (odds ratio=3.73; 95% confidence interval [CI], 1.98-7.04; P<0.001) regardless of which cutoff value was selected for CD38 and ZAP-70.

In comparison with IGHV mutation status, FCRL2 had the highest sensitivity, specificity, positive predictive value (PPV), and negative predictive value (NPV) among FCRL family members (Table 4). Strikingly, FCRL2 demonstrated 94.4% concordance with IGHV mutation status compared with 76.6% for CD38 and 80.4% for ZAP-70 by T/B-CLL ratio analysis. Significant correlations between FCRL2 expression and ZAP-70 T/B-CLL ratio, CD38, and ZAP-70 percentage were also found. Using the Kendall tau-b to determine the degree of association, it was found that values of 0.64, −0.61, and −0.52, respectively, indicating that FCRL2 expression correlates with these markers as well. It was also confirmed that the value of CD38 (sensitivity for MT-CLL was 89.1%) and ZAP-70 T/B-CLL ratio (specificity for UM-CLL was 90.4%) for predicting IGHV mutation status. These findings indicate that the preferential overexpression of FCRL proteins by MT-CLL correlates with IGHV mutation status, and remarkably, FCRL2 had the strongest potential for stratifying UM-CLL and MT-CLL.

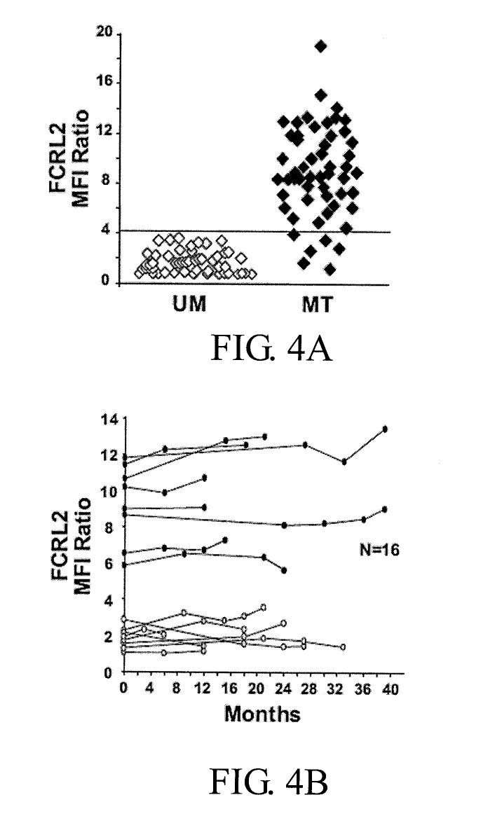

FCRL2 Expression is stable and can predict mutation status and time to first therapy. FCRL1, FCRL2, FCRL3, and FCRL5 exhibited higher surface levels of staining on MT-CLL samples compared with UM-CLL samples. Among these proteins, however, FCRL2 was distinguished as the strongest indicator of IGHV mutation status and by the magnitude of difference in its expression levels between the 2 subtypes (Table 4). The mean and median of FCRL2 staining was significantly higher among patients with MT-CLL than patients with UM-CLL at 4.7 times higher and 5.0 times higher, respectively. Using this particular anti-FCRL2 subclone (7F2) in a 2-step staining procedure, it was found that by ROC- and CART-based analyses that a cutoff MFI ratio value of 4.2 could clearly segregate patients with MT-CLL from patients with UM-CLL (FIG. 4A). Among the 107 samples however, 6 donors with MT-CLL demonstrated decreased levels of FCRL2 surface expression (MFI ratio less than 4.2) despite having IGHV regions that were less than 98% germline. Furthermore, in 4 of 6 of these patients, the expression patterns of all 4 FCRL proteins were concordant. Mutation status of the IGHV regions of these 6 samples was also reconfirmed by sequencing. Among the 52 UM-CLL samples, no discordant results were observed for FCRL2 expression.

Confirming the stability and reliability of biomarkers is essential for validating their diagnostic utility as prognostic markers and clinical tools. Among the 107 donors, 16 patients (8 with MT-CLL and 8 with UM-CLL) were analyzed at various time points over the disease course (FIG. 4B). To determine the stability of FCRL2 expression levels as a function of time, the overall concordance correlation coefficient (CCC) was calculated. Based on these 16 donors, a value of 0.931 (95% CI, 0.864-0.966) was obtained, indicating excellent stability for FCRL2.52 Sample preservation also did not appear to affect FCRL2 expression when cells were stained fresh, kept overnight at room temperature or 4° C. (as whole blood or after Ficoll), or thawed following cryopreservation. These data indicate that FCRL2 surface expression is stable over time.

To determine the clinical value of FCRL2 expression, the treatment-free interval from the time of diagnosis to initial therapy was calculated. The Cox proportional model with univariate analysis found that, similar to IGHV mutation status (hazard ratio [HR]=4.45; 95% CI, 2.47-8.00; P<0.001), FCRL2 could also predict time to first therapy (HR=4.83; 95% CI, 2.43-8.40; P<0.001). ZAP-70 T/B-CLL ratio, ZAP-70 percentage, and CD38 were also predictive of the time to first therapy with HR of 2.32 (95% CI, 1.30-4.15; P<0.004), 1.98 (95% CI, 1.16-3.38; P<0.012), and 2.11 (95% CI, 1.23-3.62; P<0.006), respectively. The log-rank test was used to calculate the median treatment-free interval for the entire cohort of 107 patients. This analysis confirmed that IGHV mutation status (P<0.001), CD38 (P<0.005), and ZAP-70 percentage (P<0.011) can all predict the time to first therapy with statistical significance (FIG. 4C). This study also found that determining ZAP-70 expression according to the T/B-CLL ratio within a sample is also predictive of the initial treatment-free interval (P<0.003; FIG. 4C). Patients with UM-CLL required treatment at a median interval of 3.51 years compared with patients with MT-CLL who first received therapy at a median interval of 13.5 years. Interestingly, all 4 FCRL proteins could predict the time to first treatment with statistical significance; however, FCRL2⁺ (MFI ratio of 4.2 or greater) patients had a median time to first therapy of 15.5 years, whereas for FCRL2″ donors, it was 3.75 years (P<0.001; FIG. 4C). The Cox model of multivariate analysis with stepwise selection found that both FCRL2 (HR=4.44; P<0.001) and clinical disease stage (HR=1.33; P<0.003) could predict clinical progression as defined by the time to first therapy, while the 9 other parameters considered in this study, including IGHV, FCRL1, FCRL3, FCRL5, CD38, ZAP-70 T/B-CLL ratio, ZAP-70 percentage, age, and sex, could not.

Considering the interaction of these variables and the clinical significance of these markers, these data indicate that FCRL2 surface expression may exhibit greater prognostic power than other FCRL proteins and presently used markers, including IGHV mutation status, CD38 expression, and ZAP-70 expression.

### Example 2

### FCRL3 Expression Correlates with the Genotype of the −169 SNP in its Promoter

The only evidence thus far for a connection between FCRL genes and autoimmunity was provided by a linkage disequilibrium analysis of 41 SNPs in the 2 Mb region encompassing the FCRL1-5 locus (Kochi et al., Nat. Genet. 37:478-85 (2005)). This extensive examination of 830 Japanese subjects with rheumatoid arthritis (RA) and 658 control subjects identified the highest association of disease susceptibility for a −169 SNP located in the FCRL3 promoter [odds ratio (OR) 2.15, P=0.00000085]. In disposed individuals this nucleotide substitution (−169T→C) results in an improved NF-κB consensus sequence binding site, increases the affinity of NF-κB for the FCRL3 promoter, and upregulates FCRL3 expression in B cells and in the synovial of RA patients. These results suggest that FCRL3 may have a pathogenic role in autoimmunity. Using the FCRL3-specific mAbs from Example 1, 40 normal subjects were genotyped for the −169 SNP and FCRL3 surface expression on CD19+B cells from PBL samples was determined. Genomic DNA from IRB consented donors was isolated and analyzed for the −169 genotype by an established pyrosequencing method (Su et al., J. Immunol. 172:7186-91 (2004)). FCRL3 surface expression was found to directly correlate with the −169C susceptibility allele in a dose dependent fashion (FIG. 4). These results demonstrate that individuals homozygous for the susceptibility allele (−169C/C) have the highest FCRL3 expression levels, followed by intermediate levels in heterozygotes (−169T/C), and lowest levels in homozygous nonsusceptible individuals (−169T/T).

1. A method of diagnosing in a subject a type of B-cell chronic lymphocytic leukemia (B-CLL) comprising the steps of:
(a) obtaining a biological sample from the subject;
(b) detecting in the biological sample expression of a first marker, wherein the first marker is FCRL3, and
(c) comparing the expression level of FCRL3 to a control FCRL3 value, a high level of FCRL3 expression compared to the control FCRL3 value indicating atypical aggressive B-CLL or typical indolent B-CLL and a low level of expression of FCRL3 as compared to the control FCRL3 value indicating typical aggressive B-CLL or atypical indolent B-CLL.

2. The method of claim 1, wherein the expression level of FCRL3 is detected by contacting the biological sample with an antibody or fragment thereof that specifically binds to the FCRL3.

3. The method of claim 2, wherein the antibody has the same epitope specificity as an antibody produced by the hybridoma cell line designated strain H3-3D2.

4. The method of claim 2, wherein the antibody is produced by the hybridoma cell line designated strain H3-3D2.

5. The method of claim 1, further comprising detecting in the biological sample expression of a second marker that distinguishes aggressive from indolent B-CLL and comparing the level of expression of the second marker with a control second marker value.

6. The method of claim 5, wherein the second marker that distinguishes aggressive from indolent B-CLL is selected from the group consisting of FCRL1, FCRL2, and FCRL5, a high level of expression of FCRL1, FCRL2, or FCRL5 as compared to the control second marker value indicating indolent B-CLL and a low level of expression of FCRL1, FCRL2, or FCRL5 as compared to the control second marker value indicating aggressive B-CLL.

7. The method of claim 6, wherein the expression level of FCRL2 is detected by contacting the biological sample with an antibody or fragment thereof that specifically binds to the FCRL2.

8. The method of claim 7, wherein the antibody has the same epitope specificity as an antibody produced by the hybridoma cell line designated strain H2-7F2.

9. The method of claim 7, wherein the antibody is produced by the hybridoma cell line designated strain H2-7F2.

10. The method of claim 6, wherein the expression level of FCRL5 is detected by contacting the biological sample with an antibody or fragment thereof that specifically binds to the FCRL5.

11. The method of claim 10, wherein the antibody has the same epitope specificity as an antibody produced by the hybridoma cell line designated strain H5-2B4.

12. The method of claim 10, wherein the antibody is produced by the hybridoma cell line designated strain H5-2B4.

13. The method of claim 1, further comprising detecting in the biological sample expression of a second marker that distinguishes typical from atypical B-CLL and comparing the level of expression of the second marker with a control second marker value.

14. The method of claim 13, wherein the second marker that distinguishes typical from atypical B-CLL is CD5, a high level of expression of CD5 as compared to the control second marker value indicating typical B-CLL and a low level of expression of CD5 as compared to the control second marker value indicating atypical B-CLL.

15. The method of claim 1, further comprising detecting in the biological sample expression of a second marker that distinguishes typical indolent B-CLL from typical aggressive B-CLL and comparing expression of the second marker to a control second marker value.

16. The method of claim 15, wherein the second marker that distinguishes typical indolent B-CLL from typical aggressive B-CLL is CD38, a high level of CD38 expression as compared to the control second marker value indicating typical aggressive B-CLL and a low level of CD38 expression as compared to the control second marker value indicating typical indolent B-CLL.

17. The method of claim 6, further comprising detecting in the biological sample expression of a third marker and comparing the level of the third marker with a control third marker value, wherein the third marker is selected from the group consisting of CDS, CD38, or both CD5 and CD38.

18. The method of claim 1, further comprising detecting ZAP70, CD38, or any combination of ZAP70, and CD38.

19. The method of claim 18, wherein a high level of ZAP-70 expression as compared to a control indicating aggressive B-CLL and a low level of ZAP-70 expression as compared to the control indicating indolent B-CLL.

20. The method of claim 1, further comprising detecting IgV_{H} mutational status.

21. A method of diagnosing in a subject a type of B-cell chronic lymphocytic leukemia (B-CLL) comprising the steps of:
(a) obtaining a biological sample from the subject;
(b) detecting in the biological sample expression of a first marker, wherein the first marker is FCRL2, and
(c) comparing the expression level of FCRL2 to a control FCRL2 value, a high level of FCRL2 expression compared to the control FCRL2 value indicating indolent B-CLL and a low level of expression of FCRL2 as compared to the control FCRL2 value indicating aggressive B-CLL.

22. The method of claim 21, wherein the expression level of FCRL2 is detected by contacting the biological sample with an antibody or fragment thereof that specifically binds to the FCRL2.

23. The method of claim 21, wherein the antibody has the same epitope specificity as an antibody produced by the hybridoma cell line designated strain H2-7F2.

24. The method of claim 21, wherein the antibody is produced by the hybridoma cell line designated strain H2-7F2.

25. The method of claim 21, further comprising detecting in the biological sample expression of a second marker that distinguishes typical from atypical B-CLL and comparing the level of expression of the second marker with a control second marker value.

26. The method of claim 25, wherein the second marker that distinguishes typical from atypical B-CLL is CD5, a high level of expression of CD5 as compared to the control second marker value indicating typical B-CLL and a low level of expression of CD5 as compared to the control second marker value indicating atypical B-CLL.

27. The method of claim 21, further comprising detecting in the biological sample expression of a second marker that distinguishes aggressive from indolent B-CLL and comparing the level of expression of the second marker with a control second marker value.

28. The method of claim 27, wherein the second marker is ZAP70, CD38, or both ZAP70 and CD38.

29. The method of claim 28, wherein a high level of ZAP70 expression as compared to the control second marker value indicating aggressive B-CLL and a low level of ZAP70 expression as compared to the control second marker value indicating indolent B-CLL.

30. The method of claim 28, wherein a high level of CD38 expression as compared to the control second marker value indicating aggressive B-CLL and a low level of ZAP70 expression as compared to the control second marker value indicating indolent B-CLL.

31. The method of claim 21, further comprising detecting IgV_{H} mutational status.

32. An antibody having the same epitope specificity as an antibody produced by the hybridoma cell line designated strain H3-3D2.

33. The antibody of claim 20, wherein the antibody is produced by the hybridoma cell line designated strain H3-3D2.

34. An antibody having the same epitope specificity as an antibody produced by the hybridoma cell line designated strain H2-7F2.

35. The antibody of claim 22, wherein the antibody is produced by the hybridoma cell line designated strain H2-7F2.

36. An antibody having the same epitope specificity as an antibody produced by the hybridoma cell line designated strain H5-2B4.

37. The antibody of claim 24, wherein the antibody is produced by the hybridoma cell line designated strain H5-2B4.

38. A humanized version or fragment of the antibody of claim 32, wherein the humanized version or fragment specifically binds FcRL 3.

39. (canceled)

40. A method of treating a subject with a B-CLL comprising administering to the subject an effective amount of the antibody of claim 32 or a fragment or humanized version thereof.

41. The method of claim 40, wherein the B-CLL is indolent B-CLL.

42. The method of claim 40 wherein the B-CLL is atypical indolent B-CLL.

43. A humanized version or fragment of the antibody of claim 34, wherein the humanized version or fragment specifically binds FcRL 2.

44. A humanized version or fragment of the antibody of claim 36, wherein the humanized version or fragment specifically binds FcRL 5.


SEQUENCE LISTING (2 sequences)

SEQ ID NO: 1 - protein, 19 aa, Artificial Sequence
  Consensus sequence
XXXYXXXXXXXXXXXYXXX

SEQ ID NO: 2 - protein, 6 aa, Artificial Sequence
  Consensus sequence
XXYXXX